(19) United States
(12) Patent Application Publication
(10) Pub. No.: US 2025/0099426 A1
(43) Pub. Date: Mar. 27, 2025

(54) Indolium Compounds for Treating Cancer

(71) Applicant: Emory University, Atlanta, GA (US)

(72) Inventor: Jack L. Arbiser, Atlanta, GA (US)

(21) Appl. No.: 18/853,643

(22) PCT Filed: Mar. 28, 2023
(86) PCT No.: PCT/US2023/016582

§ 371 Date: Oct. 2, 2024

Related U.S. Application Data
(60) Provisional application No. 63/326,204, filed on Mar. 31, 2022.

(51) Int. Cl. A61K 31/4045; A61K 45/06; A61P 35/00

(52) U.S. Cl. CPC A61K 31/4045; A61K 45/06; A61P 35/00

(57) ABSTRACT

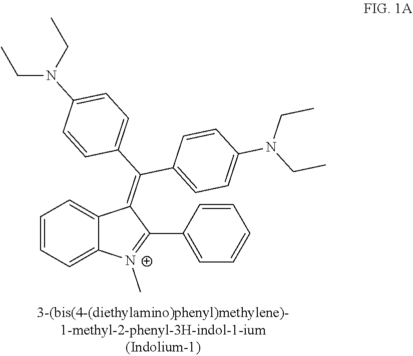 [SMILES: CCN(CC)c1ccc(C(=C2C(c3ccccc3)=[N+](C)c3ccccc32)c2ccc(N(CC)CC)cc2)cc1]

This disclosure relates to compounds, compositions, and methods for managing cancer treatment using Indolium compounds and salts thereof as disclosed herein. In certain embodiments, this disclosure relates to treating or preventing cancer comprising administering an effective amount 3-(bis(4-(diethylamino)phenyl) methylene)-1-methyl-2-phenyl-3H-indol-1-ium (Indolium-1), salt or derivative thereof to a subject in need thereof, optionally in combination with another anticancer agent. In certain embodiments, the cancer is a melanoma. In certain embodiments, this disclosure relates to pharmaceutical compositions having Indolium compounds and salts thereof.

### CROSS-REFERENCE TO RELATED APPLICATIONS

This application claims the benefit of U.S. Provisional Application No. 63/326,204 filed Mar. 31, 2022. The entirety of this application is hereby incorporated by reference for all purposes.

### BACKGROUND

Melanoma remains the major cause of death due to a skin cancer. During the past two decades, there has been a major change in the treatment of patients with melanoma. The majority of cutaneous melanomas demonstrate expression of mutant BRAF and have been targeted by BRAF and MEK inhibitors. Resistance has developed to these inhibitors through a wide variety of mechanisms, with both intrinsic resistance in existing tumors and developments of mutations (extrinsic resistance). Other subsets of melanoma benefit less from targeted therapies, e.g., uveal melanoma, acral melanoma, and melanomas mutant in NRAS and triple negative melanomas (harboring wild type BRAF, NRAS and PTEN).

Immunotherapies have played a major role in the treatment of patients with melanoma. In some instances, long term remission has been achieved. However, mixed response and relapse is not uncommon. Tumors which are responsive to immunotherapy in general have a high mutational burden which is likely a source of neoantigens. They also express PDL1, PD1, and CTLA4, although an immunohistochemical test which predicts response to immunotherapy has proven elusive. Finally, both intrinsic resistance due to tumor heterogeneity, and extrinsic resistance, due to loss of neoantigens or de-differentiation has been observed. Thus, there is a need to identify improved treatment options.

Arbiser et al. report combination therapy of imiquimod and gentian violet for cutaneous melanoma metastases. Journal of the American Academy of Dermatology 2012, 67, e81-83. See also US Patent No. U.S. Pat. No. 10,376,522 (2019).

Guinot et al. report cyclic terminal groups in di- and tri-arylmethane dyes. J. Chem. Soc., Perkin Trans. 2, 1998, 297-303. See also WO2012/128318 (page 115) and DE2625944 (1977).

References cited herein are not an admission of prior art.

### SUMMARY

This disclosure relates to compounds, compositions, and methods for managing cancer treatment using Indolium compounds and salts thereof. In certain embodiments, this disclosure relates to treating or preventing cancer comprising administering an effective amount 3-(bis(4-(diethylamino)phenyl) methylene)-1-methyl-2-phenyl-3H-indol-1-ium (Indolium-1), salt or derivative thereof to a subject in need thereof, optionally in combination with another anticancer agent. In certain embodiments, the cancer is a melanoma.

In certain embodiments, the Indolium compound is a compound having the following formula:

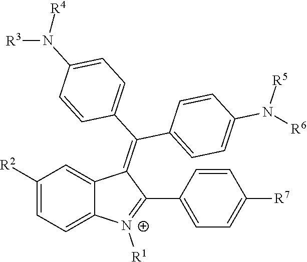

or salt thereof wherein,
R¹, R², R³, R⁴, R⁵, R⁶, and R⁷, are individually and independently selected from hydrogen, halogen, alkyl, hydroxy, alkoxy, mercapto, alkylthio, amino, alkylamino, dialkylamino, carboxyl, nitro, and nitrile.

In certain embodiments, the cancer is melanoma, uveal melanoma, acral melanoma, melanoma mutant in NRAS, or triple negative melanoma. In certain embodiments, the melanoma has become resistant to an inhibitor of v-Raf murine sarcoma viral oncogene homolog B (BRAF) and/or an inhibitor of mitogen-activated protein kinase kinase (MEK).

In certain embodiments, the anticancer agent is an inhibitor of v-Raf murine sarcoma viral oncogene homolog B (BRAF) such as vemurafenib, dabrafenib, and encorafenib.

In certain embodiments, the anticancer agent is an inhibitor of mitogen-activated protein kinase kinase (MEK) such as trametinib, binimetinib, selumetinib, and cobimetinib.

In certain embodiments, the anticancer agent is an inhibitor of or an inhibitor of protein kinase B (AKT) such as ipatasertib.

In certain embodiments, the anticancer agent is an anti-CTLA4 antibody such as ipilimumab and tremelimumab.

In certain embodiments, the anticancer agent is an anti-PD1 antibody such as nivolumab, pembrolizumab, and cemiplimab.

In certain embodiments, the anticancer agent is an anti-PD-L1 antibody such as atezolizumab, avelumab, and durvalumab.

In certain embodiments, the Indolium compound is administered in combination with T cells expressing a CAR.

In certain embodiments, the cancer is selected from bladder cancer, lung cancer, breast cancer, colon cancer, rectal cancer, endometrial cancer, pancreatic cancer, kidney cancer, prostate cancer, thyroid cancer, brain cancer, multiple myeloma, lymphoma, or leukemia.

In certain embodiments, this disclosure relates to pharmaceutical compositions comprising a compound having the following formula:

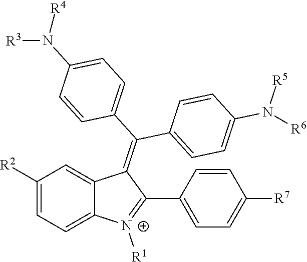

or salt wherein,
R¹, R², R³, R⁴, R⁵, R⁶, and R⁷, are individually and independently selected from hydrogen, halogen, alkyl, hydroxy, alkoxy, mercapto, alkylthio, amino, alkylamino, dialkylamino, carboxyl, nitro, and nitrile, and a pharmaceutically acceptable excipient.

In certain embodiments, the compound is an optionally substituted 3-(bis(4-(diethylamino) phenyl) methylene)-1-methyl-2-phenyl-3H-indol-1-ium (Indolium-1) or salt thereof salt.

In certain embodiments, the pharmaceutical composition is in the form of a lotion, gel, cream, pill, tablet, capsule, or gel capsule. In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiments, this disclosure relates to the use of an Indolium compound, salt, or pharmaceutical composition as disclosed herein for use in treating cancer. In certain embodiments, this disclosure relates to the production of a medicament comprising an Indolium compound, salt, or pharmaceutical composition as disclosed herein for use in treating cancer

### BRIEF DESCRIPTION OF THE SEVERAL VIEWS OF THE DRAWINGS

FIG. 1A illustrates the chemical structure and name of Indolium-1 [3-(bis(4-(diethylamino) phenyl) methylene)-1-methyl-2-phenyl-3H-indol-1-ium.

FIG. 1B illustrates a synthetic method for the preparation of Indolium-1.

FIG. 2 shows data on tumor growth in control versus Indolium 1 treated mice. Indolium 1 treatment results in reduced tumor volume over 4 weeks. Mice were inoculated with LM36R, a vemurafenib-resistant melanoma cell line, at 1×10⁶ cells/mouse. Mice were then treated with Indolium via IP administration three times a week at a concentration of 3 mg/kg/week. Tumor growth was compared to control vehicle (sterile water). Results indicate a possible reduction in tumor growth over period of observance. (N=5/group, p-value=0.03) at week 4.

FIG. 3 shows data on the relative cell viability of LM36R cells treated with certain compounds. Lanes: 1) Control 2) Indolium 1 (1 μM) 3) MEK Inhibitor U0126 (10 μM) 4) Akt Inhibitor II (10 μM) 5) Indolium 1 (1 μM)+MEK Inhibitor U0126 (10 μM) 6) Indolium 1+Akt Inhibitor II (10 μM). *=Statistically lower cell viability compared to control (p<0.05). **=Statistically lower cell viability compared to control and statistically higher cell viability than Indolium 1 treatment (lane 2) alone. Inhibition of MAPK and Akt reverses cell death caused by Indolium 1.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” is an example, and not necessarily limited to such example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

“Subject” refers to any animal, preferably a human patient, livestock, rodent, monkey, or domestic pet.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

A “chemotherapy agent,” “chemotherapeutic,” “anti-cancer agent” or the like, refer to molecules that are recognized to aid in the treatment of a cancer. Contemplated examples include the following molecules or derivatives such as temozolomide, carmustine, bevacizumab, procarbazine, lomustine, vincristine, gefitinib, erlotinib, cisplatin, carboplatin, oxaliplatin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, paclitaxel, taxol, docetaxel, etoposide, teniposide, amsacrine, topotecan, camptothecin, bortezomib, anagrelide, tamoxifen, toremifene, raloxifene, droloxifene, idoxifene, fulvestrant, bicalutamide, flutamide, nilutamide, cyproterone, goserelin, leuprorelin, buserelin, megestrol, anastrozole, letrozole, vorozole, exemestane, finasteride, marimastat, trastuzumab, cetuximab, dasatinib, imatinib, combretastatin, thalidomide, azacitidine, azathioprine, capecitabine, chlorambucil, cyclophosphamide, cytarabine, daunorubicin, doxifluridine, epothilone, irinotecan, mechlorethamine, mercaptopurine, mitoxantrone, pemetrexed, tioguanine, valrubicin and/or lenalidomide or combinations thereof such as cyclophosphamide, methotrexate, 5-fluorouracil (CMF); doxorubicin, cyclophosphamide (AC); mustine, vincristine, procarbazine, prednisolone (MOPP); adriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

As used herein, a “chimeric antigen receptor” or “CAR” refers to a protein receptor, which introduces an antigen specificity, via an antigen binding domain, onto cells to which it is expressed (for example T cells such as naive T cells, central memory T cells, effector memory T cells or combination thereof) thus combining the antigen binding properties of the antigen binding domain with the T cell activity (e.g. lytic capacity and self-renewal) of T cells. A CAR typically includes an extracellular antigen-binding domain (ectodomain), e.g., specifically binding CD19 or BCMA, a transmembrane domain and an intracellular signaling domain. The intracellular signaling domain generally contains at least one immunoreceptor tyrosine-based activation motif (ITAM) signaling domain, e.g., derived from CD3zeta, and optionally at least one costimulatory signaling domain, e.g., derived from CD28 or 4-1BB.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing a oxygen atom with a sulfur atom, replacing an amino group with a hydroxyl group, replacing a nitrogen with a protonated carbon (CH) in an aromatic ring, replacing a bridging amino group (—NH—) with an oxy group (—O—), or vice versa. The derivative may be a prodrug. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)2Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Typical prodrugs are pharmaceutically acceptable esters. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate, and benzoate derivatives of an alcohol or acetamide, formamide and benzamide derivatives of an amine functional group in the active compound and the like.

### Methods of Use

This disclosure relates to compounds, compositions, and methods for managing cancer treatment using Indolium compounds and salts disclosed herein. In certain embodiments, this disclosure relates to treating or preventing cancer comprising administering an effective amount 3-(bis(4-(diethylamino)phenyl) methylene)-1-methyl-2-phenyl-3H-indol-1-ium (Indolium-1), salt or derivative thereof to a subject in need thereof. In certain embodiments, the cancer is a melanoma.

In certain embodiments, the Indolium compound is a compound having the following formula:

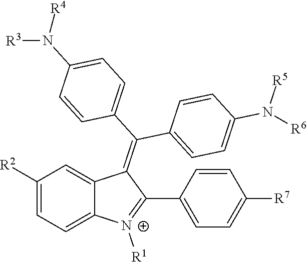

or salt thereof wherein,
R¹, R², R³, R¹, R⁵, R⁶, and R⁷, are individually and independently selected from hydrogen, halogen, alkyl, hydroxy, alkoxy, mercapto, alkylthio, amino, alkylamino, dialkylamino, carboxyl, nitro, and nitrile.

In certain embodiments, the compound is administered in combination with another anticancer agent.

In certain embodiments, the cancer is melanoma, uveal melanoma, acral melanoma, melanoma mutant in NRAS, or triple negative melanoma. In certain embodiments, the melanoma has become resistant to an inhibitor of v-Raf murine sarcoma viral oncogene homolog B (BRAF) and/or an inhibitor of mitogen-activated protein kinase kinase (MEK).

In certain embodiments, the anticancer agent is an inhibitor of v-Raf murine sarcoma viral oncogene homolog B (BRAF) such as vemurafenib, dabrafenib, and encorafenib.

In certain embodiments, the anticancer agent is an inhibitor of mitogen-activated protein kinase kinase (MEK) such as trametinib, binimetinib, selumetinib, and cobimetinib.

In certain embodiments, the anticancer agent is an inhibitor of or an inhibitor of protein kinase B (AKT) such as ipatasertib.

In certain embodiments, this disclosure contemplates the use of BRAF/MEK inhibitors with an Indolium compound in a sequential fashion. In certain embodiments, methods comprise administering a BRAF and/or MEK inhibitor, thereafter, ceasing administering a BRAF and/or MEK inhibitor, e.g., such that the BRAF and/or MEK inhibitor is no longer bioavailable, and thereafter administering an Indolium compound disclosed herein.

In certain embodiments, this disclosure contemplates the use of an Indolium compound disclosed herein with a BRAF/MEK inhibitor in a sequential fashion. In certain embodiments, methods comprise administering an Indolium compound disclosed herein, thereafter ceasing administering an Indolium compound disclosed herein, e.g., such that the Indolium compound disclosed herein is no longer bioavailable, and thereafter administering a BRAF and/or MEK inhibitor.

In certain embodiments, the anticancer agent is an anti-CTLA4 antibody such as ipilimumab and tremelimumab.

In certain embodiments, the anticancer agent is an anti-PD1 antibody such as nivolumab, pembrolizumab, and cemiplimab.

In certain embodiments, the anticancer agent is an anti-PD-L1 antibody such as atezolizumab, avelumab, and durvalumab.

In certain embodiments, the anticancer agent is a T cell therapy comprising a CAR.

In certain embodiments, the cancer is selected from bladder cancer, lung cancer, breast cancer, colon cancer, rectal cancer, endometrial cancer, pancreatic cancer, kidney cancer, prostate cancer, thyroid cancer, brain cancer, multiple myeloma, lymphoma, or leukemia.

In certain embodiments, this disclosure relates to methods of treating cancer comprising administering an effective amount of Indolium compounds disclosed herein to a subject in need thereof. In certain embodiments, the subject is suffering from a neoplasm. In certain embodiments, the neoplasm has a mutation encoding a V600E amino acid substitution present in the coding sequence for BRAF or other mutation. In certain embodiments, the subject is suffering from metastatic melanoma.

In certain embodiments, the subject is diagnosed with cancer or hematological malignancy. In certain embodiments, the hematological malignancy is multiple myeloma, leukemia, or lymphoma. In certain embodiments, the hematological malignancy is acute lymphoblastic leukemia (ALL), acute myelogenous leukemia (AML), chronic lymphocytic leukemia (CLL), small lymphocytic lymphoma (SLL), chronic myelogenous leukemia, acute monocytic leukemia (AMOL), Hodgkin's lymphomas, and non-Hodgkin's lymphomas such as Burkitt lymphoma, B-cell lymphoma.

In certain embodiments, the Indolium compounds disclosed herein are administered in combination with a second anticancer agent. In certain embodiments, the second therapeutic agent is an anti-cancer agent. In certain embodiments, the second therapeutic agent is a BRAF inhibitor. In certain embodiments, the BRAF inhibitor is vemurafenib, sorafenib, encorafenib, and dabrafenib.

Accordingly, Indolium compounds disclosed herein may be used in the treatment of a neoplasm, particularly a susceptible neoplasm (a cancer or tumor) in a mammal. The present disclosure also provides a method for treating a neoplasm, particularly a susceptible neoplasm in a mammal in need thereof, which method comprises administering to the mammal a therapeutically effective amount of the compound disclosed herein. In certain embodiments, the disclosure also provides the use of Indolium compounds disclosed herein for the preparation of a medicament for the treatment of neoplasm, particularly a susceptible neoplasm, in a mammal.

In certain embodiment, this disclosure relates to methods for the treatment a subject at risk of, exhibiting symptoms of, suspected of, or diagnosed with a cancer or neoplasm selected from skin cancer, melanoma, Barret's adenocarcinoma; biliary tract carcinomas; breast cancer; cervical cancer; cholangiocarcinoma; central nervous system tumors including primary CNS tumors such as glioblastomas, astrocytomas (including glioblastoma multiforme) and ependymomas, and secondary CNS tumors (i.e., metastases to the central nervous system of tumors originating outside of the central nervous system), colorectal cancer, including large intestinal colon carcinoma; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer and non-small cell lung cancer; ovarian cancer; endometrial cancer; pancreatic cancer; pituitary adenoma; prostate cancer; renal cancer; sarcoma; and thyroid cancers.

In certain embodiments, this disclosure relates to the use of Indolium compounds disclosed herein for the preparation of a medicament for the treatment of Barret's adenocarcinoma; billiary tract carcinomas; breast cancer; cervical cancer; cholangiocarcinoma; central nervous system tumors including primary CNS tumors such as glioblastomas, astrocytomas (e.g., glioblastoma multiforme) and ependymomas, and secondary CNS tumors (i.e., metastases to the central nervous system of tumors originating outside of the central nervous system); colorectal cancer including large intestinal colon carcinoma; gastric cancer; carcinoma of the head and neck including squamous cell carcinoma of the head and neck; hematologic cancers including leukemias and lymphomas such as acute lymphoblastic leukemia, acute myelogenous leukemia (AML), myelodysplastic syndromes, chronic myelogenous leukemia, Hodgkin's lymphoma, non-Hodgkin's lymphoma, megakaryoblastic leukemia, multiple myeloma and erythroleukemia; hepatocellular carcinoma; lung cancer including small cell lung cancer and non-small cell lung cancer; ovarian cancer; endometrial cancer; pancreatic cancer; pituitary adenoma; prostate cancer; renal cancer; sarcoma; skin cancers including melanomas; and thyroid cancers, or any subset thereof, in a mammal (e.g., human).

The Indolium compounds disclosed herein can be used alone in the treatment of each of the foregoing conditions or can be used to provide additive or potentially synergistic effects with certain existing chemotherapies, radiation, biological or immunotherapeutics (including monoclonal antibodies) and vaccines. The Indolium compounds disclosed herein may be useful for restoring effectiveness of certain existing chemotherapies and radiation and or increasing sensitivity to certain existing chemotherapies and/or radiation.

In certain embodiments, this disclosure provides a method for treating a susceptible neoplasm in a mammal in need thereof comprising the steps of: (a) analyzing a sample from said neoplasm to determine whether an activating mutation is present in the coding sequence for BRAF in cells of said neoplasm; (b) selecting a mammal having a neoplasm with an activating mutation in the coding sequence for BRAF; and (c) administering a therapeutically effective amount of an Indolium compound disclosed herein optionally in combination with a BRAF inhibitor to the mammal selected in step (b).

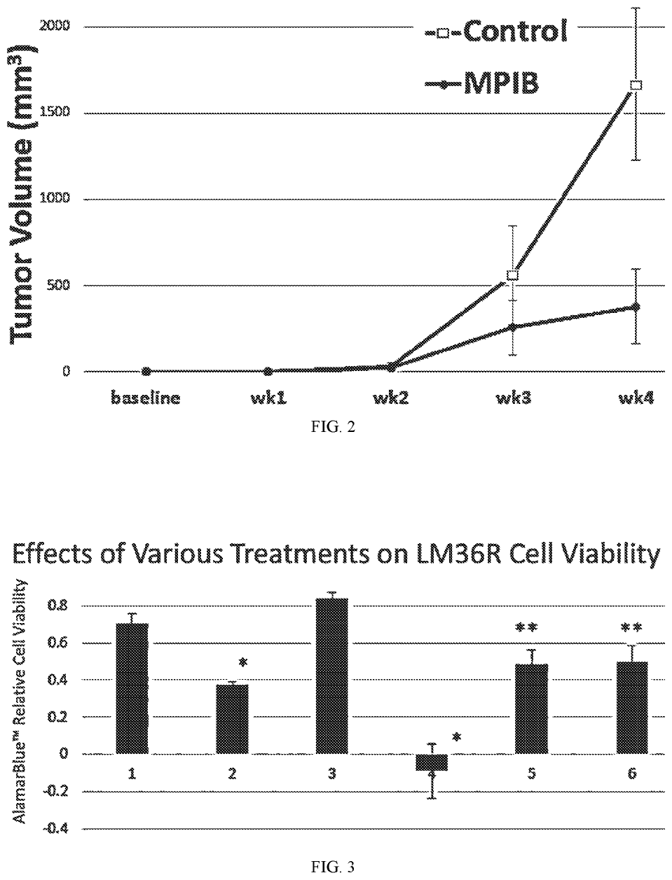

In certain embodiments, the activating mutation present in the coding sequence for BRAF results in a BRAF having an amino acid substitution selected from the group consisting of R462I, I463S, G464V, G464E, G466A, G466E, G466V, G469A, G469E, D594V, F595L, G596R, L597V, L597R, T5991, V600E, V600D, V600K, V600R, T119S, and K601E. See, for example, FIG. 2 of Halilovic and Solvit (2008) Current Opinion in Pharmacology 8:419-26.

In certain embodiments, this disclosure relates to a method for treating a susceptible neoplasm in a mammal in need thereof comprising the steps of: (a) analyzing a sample from said neoplasm to determine whether a mutation encoding a V600E, V600D or V600R amino acid substitution is present in the coding sequence for BRAF in cells of said neoplasm; (b) selecting a mammal having a neoplasm with a mutation encoding the V600E, V600D or V600R amino acid substitution in BRAF; and (c) administering a therapeutically effective amount of an Indolium compound disclosed herein optionally in combination with a BRAF inhibitor to the mammal selected in step (b).

The V600E amino acid substitution in BRAF is described, for example, in Kumar et al. (2004) J Invest Dermatol. 122(2):342-8. This mutation commonly results from a T1799A mutation in the coding sequence for human BRAF. Accordingly, in one embodiment of the present disclosure, the step of analyzing a sample from said neoplasm to determine whether a mutation encoding a V600E amino acid substitution is present in the coding sequence for BRAF is performed by determining whether the coding sequence for BRAF in cells of the neoplasm contains the T1799A mutation.

The sample of the neoplasm to be analyzed for the presence of BRAF activating mutations can be derived from a variety of sources including, but not limited to, single cells, a collection of cells, tissue, cell culture, bone marrow, blood, or other bodily fluids. The tissue or cell source may include a tissue biopsy sample, a cell sorted population, cell culture, or a single cell. In selecting a sample, the percentage of the sample that constitutes neoplastic cells should be considered. In some embodiments, the sample from the neoplasm is fixed using a preservative prior to analyzing for the presence of an activating mutation.

The step of analyzing a sample from the neoplasm to determine whether an activating mutation is present in the coding sequence for BRAF in cells of said neoplasm may be performed using any method known in the art. For example, the coding sequence for BRAF in cells of the sample may be analyzed to determine if it contains a mutation which results in the expression of activated BRAF. Methods for detecting such mutations are well known in the art. See, for example, Whitcombe et al. (1999) Nature Biotechnology 17:804-7, Gibson (2006) Clinica Chimica Acta 363: 32-47, Kim and Misra (2007) Annual Review of Biomedical Engineering 9:289-320, and U.S. Pat. Nos. 6,326,145 and 6,270,967). Alternatively, activating mutations in BRAF may be identified by directly detecting the activated BRAF protein using an agent (e.g., an antibody) that selectively binds activated BRAF.

The precise therapeutically effective amount of an Indolium compound disclosed herein will depend on a number of factors. These variables determine what dose of compound needs to be administered in a sufficient percentage and for a sufficient amount of time to have the desired effect on the condition being treated (e.g., neoplasm). The amount of compound administered will also depend on factors related to patients and disease including, but not limited to, the following: the age, weight, concomitant medications, and medical condition of the subject being treated, the precise condition requiring treatment and its severity, the nature of the formulation, and the route of administration. Ultimately, the dose will be at the discretion of the attendant physician or veterinarian. Typically, the compound disclosed herein will be given for treatment in the range of 0.01 to 30 mg/kg body weight of recipient (mammal) per day or per dose or per cycle of treatment and more usually in the range of 0.1 to 10 mg/kg body weight per day or per dose or per cycle of treatment. Thus, for an adult human being treated for a condition, the actual amount per day or per dose or per cycle of treatment would usually be from 1 to 2000 mg and this amount may be given in a single or multiple doses per day or per dose or per cycle of treatment. The full spectrum of dosing regimens may be employed ranging from continuous dosing (with daily doses) to intermittent dosing.

In the above-described methods of treatment and uses, Indolium compounds herein may be employed alone, in combination with one or more other compounds disclosed herein or in combination with other therapeutic methods or agents. In particular, in methods of treating a condition attenuated by combination with other chemotherapeutic, biologic, hormonal, antibody and supportive care agents is envisaged as well as combination with surgical therapy and radiotherapy. Supportive care agents include analgesics, anti-emetics and agents used to treat hematologic side effects such as neutropenia. Analgesics are well known in the art. Anti-emetics include but are not limited to 5HT3 antagonists such as ondansetron, granisetron, dolasetron, palonosetron and the like; prochlorperazine; metoclopramide; diphenhydramine; promethazine; dexamethasone; lorazepam; haloperidol; dronabinol; olanzapine; and neurokinin-1 antagonists such as aprepitant, fosaprepitant and casopitant administered alone or in various combinations.

Combination therapies according to the disclosure thus comprise the administration of Indolium compounds disclosed herein and the use of at least one other treatment method. In one embodiment, combination therapies according to the disclosure comprise the administration of Indolium compounds disclosed herein and surgical therapy. In one embodiment, combination therapies according to the disclosure comprise the administration of Indolium compounds disclosed herein and radiotherapy. In one embodiment, combination therapies according to the disclosure comprise the administration of Indolium compounds disclosed herein and at least one supportive care agent (e.g., at least one anti-emetic agent). In one embodiment, combination therapies according to the present disclosure comprise the administration of Indolium compounds disclosed herein and at least one other chemotherapeutic agent. In one particular embodiment, the disclosure comprises the administration of Indolium compounds disclosed herein and at least one anti-neoplastic agent.

As an additional aspect, the present disclosure provides the methods of treatment and uses as described above, which comprise administering Indolium compounds disclosed herein together with at least one chemotherapeutic agent. In one particular embodiment, the chemotherapeutic agent is an anti-neoplastic agent.

The disclosure also provides methods of treatment and uses as described above, which comprise administering Indolium compounds disclosed herein together with at least one supportive care agent (e.g., anti-emetic agent).

The Indolium compounds disclosed herein and at least one additional anti-neoplastic or supportive care therapy may be employed in combination concomitantly or sequentially in any therapeutically appropriate combination. The administration of Indolium compounds disclosed herein with one or more other anti-neoplastic agents may be in combination in accordance with the disclosure by administration concomitantly in one unitary pharmaceutical composition including both or all compounds or two or more separate pharmaceutical compositions each including one or more of the compounds. The components of the combination may be administered separately in a sequential manner wherein one active ingredient is administered first and the other(s) second or vice versa. Such sequential administration may be close in time or remote in time.

Typically, any chemotherapeutic agent that has activity against a susceptible neoplasm being treated may be utilized in combination with Indolium compounds herein, provided that the particular agent is clinically compatible with therapy employing a compound disclosed herein. Typical anti-neoplastic agents useful in the present disclosure include, but are not limited to: alkylating agents, anti-metabolites, antitumor antibiotics, antimitotic agents, topoisomerase I and II inhibitors, hormones and hormonal analogues; retinoids, signal transduction pathway inhibitors including inhibitors of cell growth or growth factor function, angiogenesis inhibitors, and serine/threonine or other kinase inhibitors; cyclin dependent kinase inhibitors; antisense therapies and immunotherapeutic agents, including monoclonals, vaccines or other biological agents.

Alkylating agents are non-phase specific anti-neoplastic agents and strong electrophiles. Typically, alkylating agents form covalent linkages, by alkylation, to DNA through nucleophilic moieties of the DNA molecule such as phosphate, amino, and hydroxyl groups. Such alkylation disrupts nucleic acid function leading to cell death. Alkylating agents may be employed in combination with the Indolium compounds disclosed herein in the compositions and methods described above. Examples of alkylating agents include but are not limited to nitrogen mustards such as cyclophosphamides, temozolomide, melphalan, and chlorambucil; oxazaphosphorines; alkyl sulfonates such as busulfan; nitrosoureas such as carmustine; triazenes such as dacarbazine; and platinum coordination complexes such as cisplatin, oxaliplatin and carboplatin.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. The end result of discontinuing S phase is cell death. Antimetabolite neoplastic agents may be employed in combination with the

Indolium compounds disclosed herein in the compositions and methods described above. Examples of antimetabolite anti-neoplastic agents include but are not limited to purine and pyrimidine analogues and anti-folate compounds, and more specifically, hydroxyurea, cytosine, arabinoside, raltitrexed, tegafur, fluorouracil (e.g., 5FU), methotrexate, cytarabine, mercaptopurine and thioguanine.

Antitumor antibiotic agents are non-phase specific agents, which bind to or intercalate with DNA. Typically, such action disrupts ordinary function of the nucleic acids, leading to cell death. Antitumor antibiotics may be employed in combination with the Indolium compounds disclosed herein in the compositions and methods described above. Examples of antitumor antibiotic agents include, but are not limited to, actinomycins such as dactinomycin; anthracyclines such as daunorubicin, doxorubicin, idarubicin, epirubicin and mitoxantrone; mitomycin C and bleomycins.

Antimicrotubule or antimitotic agents are phase specific agents active against the microtubules of tumor cells during M or the mitosis phase of the cell cycle. Antimitotic agents may be employed in combination with the Indolium compounds disclosed herein in the compositions and methods described above. Examples of antimitotic agents include, but are not limited to, diterpenoids, vinca alkaloids, polo-like kinase (Plk) inhibitors and CenpE inhibitors. Examples of diterpenoids include, but are not limited to, paclitaxel and its analog docetaxel. Examples of vinca alkaloids include, but are not limited to, vinblastine, vincristine, vindesine and vinorelbine.

Topoisomerase inhibitors include inhibitors of Topoisomerase II and inhibitors of Topoisomerase I. Topoisomerase II inhibitors, such as epipodophyllotoxins, are anti-neoplastic agents derived from the mandrake plant, that typically affect cells in the S and G2 phases of the cell cycle by forming a ternary complex with topoisomerase II and DNA, causing DNA strand breaks. The strand breaks accumulate, and cell death follows. Examples of epipodophyllotoxins include, but are not limited to, etoposide and teniposide. Camptothecins, including camptothecin and camptothecin derivatives, are available or under development as Topoisomerase I inhibitors. Examples of camptothecins include, but are not limited to amsacrine, irinotecan, topotecan, and the various optical forms of 7-(4-methylpiperazino-methylene)-10,11-ethylenedioxy-20-camptothecin. Topoisomerase inhibitors may be employed in combination with the Indolium compounds disclosed herein in the compositions and methods described above.

Hormones and hormonal analogues are useful compounds for treating cancers in which there is a relationship between the hormone(s) and growth and/or lack of growth of the cancer. Antitumor hormones and hormonal analogues may be employed in combination with the Indolium compounds disclosed herein in the compositions and methods described above. Examples of hormones and hormonal analogues believed to be useful in the treatment of neoplasms include, but are not limited to antiestrogens, such as tamoxifen, toremifene, raloxifene, fulvestrant, idoxifene and droloxifene; anti-androgens; such as flutamide, nilutamide, bicalutamide and cyproterone acetate; adrenocorticosteroids such as prednisone and prednisolone; aminoglutethimide and other aromatase inhibitors such as anastrozole, letrozole, vorozole, and exemestane; megestrol acetate; 5alpha-reductase inhibitors such as finasteride and dutasteride; and gonadotropin-releasing hormones (GnRH) and analogues thereof, such as Luteinizing Hormone-releasing Hormone (LHRH) agonists and antagonists such as goserelin leuprolide, leuprorelin and buserelin.

Examples of specific retinoids that may be used in combination with the Indolium compounds disclosed herein include: retinoic acid; all-trans-retinoic acid (“ATRA” also known as “tretinoin”); tamibarotene (“Am80”); 9-cis-retinoic acid ((2E,4E,6Z,8E)-3,7-Dimethyl-9-(2,6,6-trimethylcyclohex-1-enyl)nona-2,4,6,- 8-tetraenoic Acid) (also known as “9-cis-Tretinoin”) (available from Sigma); Isotretinoin ((2Z,4E,6E,8E)-3,7-dimethyl-9-(2,6,6-trimethyl-1-cyclohexenyl)nona-2,4,6,- 8-tetraenoic acid) (also known as “13-cis-retinoic acid”) (ACCUTANE™); Am580 (4-(5,6,7,8-tetrahydro-5,5,8,8-tetramethyl-2-naphtamido) benzoic acid), See, M. Gianni, Blood 1996 87(4):1520-1531; TTNPB (4-[E-2-(5,6,7,8-Tetrahydro-5,5,8,8-tetramethyl-2-naphthalenyl)-1-propeny-1]benzoic acid) (also known as “Ro 13-7410”) See, M. F. Boehm et al. J. Med. Chem. 1994 37:2930 and R. P. Bissonnette et al., Mol. Cell. Biol. 1995 15:5576; and BMS753 (4-[[(2,3-dihydro-1,1,3,3-tetramethyl-2-oxo-1H-inden-5-yl)carbonyl]amino]-benzoic acid) See, U.S. Pat. No. 6,184,256.

Several inhibitors of growth factor receptors are contemplated including ligand antagonists, antibodies, tyrosine kinase inhibitors, anti-sense oligonucleotides and aptamers. Any of these growth factor receptor inhibitors may be employed in combination with the Indolium compounds disclosed herein in any of the compositions and methods/uses described herein. Trastuzumab is an example of an anti-erbB2 antibody inhibitor of growth factor function. One example of an anti-erbB1 antibody inhibitor of growth factor function is cetuximab. Bevacizumab is an example of a monoclonal antibody directed against VEGFR. Examples of small molecule inhibitors of epidermal growth factor receptors include but are not limited to lapatinib and erlotinib. Imatinib is one example of a PDGFR inhibitor. Examples of VEGFR inhibitors include pazopanib, ZD6474, AZD2171, PTK787, sunitinib and sorafenib.

In certain embodiments, Indolium compounds disclosed herein can be used and formulated in combination with the anti-cancer agents that are PD-1 antibodies such as nivolumab, pembrolizumab, pidilizumab, atezolizumab or CTLA-4 antibodies such as ipilimumab and tremelimumab.

The pharmaceutical compositions of the present disclosure can be administered to subjects either orally, rectally, parenterally (intravenously, intramuscularly, or subcutaneously), intracistemally, intravaginally, intraperitoneally, intravesically, locally (powders, ointments, or drops), or as a buccal or nasal spray. Pharmaceutically acceptable salts, solvates and hydrates of the compounds listed are also useful in the method of the disclosure and in pharmaceutical compositions of the disclosure.

### Pharmaceutical Compositions

The Indolium compounds of the present disclosure can be administered to a subject either alone or as a part of a pharmaceutical composition. In certain embodiments, the pharmaceutical composition is in the form of a tablet, pill, capsule, gel, gel capsule or cream. In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiments, this disclosure relates to pharmaceutical compositions comprising Indolium compounds disclosed herein having the following formula:

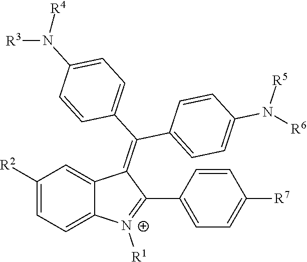

or salt wherein,
R¹, R², R³, R⁴, R⁵, R⁶, and R⁷, are individually and independently selected from hydrogen, halogen, alkyl, hydroxy, alkoxy, mercapto, alkylthio, amino, alkylamino, dialkylamino, carboxyl, nitro, and nitrile, and a pharmaceutically acceptable excipient.

In certain embodiments, the compound is an optionally substituted 3-(bis(4-(diethylamino) phenyl) methylene)-1-methyl-2-phenyl-3H-indol-1-ium (Indolium-1) or salt thereof salt.

In certain embodiments, the pharmaceutically acceptable excipient is lactose, sucrose, mannitol, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In another embodiment, the disclosure provides a pharmaceutical composition comprising Indolium compounds disclosed herein further comprising at least one other chemotherapeutic agent, more particularly, the chemotherapeutic agent is an anti-neoplastic agent.

In certain embodiments, pharmaceutical composition is in solid form surrounded by an enteric coating. In certain embodiments, the enteric coating comprises methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, or combinations thereof.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil) and injectable organic esters such as ethyl oleate. Proper fluidity can be maintained, for example, by the use of a coating such as lecithin.

These compositions may also contain adjuvants such as preserving, emulsifying, and dispensing agents. Prevention of the action of microorganisms may be controlled by addition of any of various antibacterial and antifungal agents, example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carrier) such as sodium citrate or dicalcium phosphate or: (a) fillers or extenders, as for example, starches, lactose, sucrose, glucose, mannitol and silicic acid, (b) binders, as for example, carboxymethylcellulose, alginates, gelatin, polyvinylpyrrolidone, sucrose, and acacia, (c) humectants, as for example, glycerol (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar and as high molecular weight polyethylene glycols, and the like.

Solid dosage forms such as tablets, capsules, pills, and granules can be prepared with coatings and shells, such as enteric coatings and others may contain opacifying agents so that release of the Indolium compound is in a certain part of the intestinal tract in a delayed manner. Examples of embedding compositions which can be used are polymeric substances and waxes. The active compounds can also be used in micro-encapsulated form, if appropriate, with one or more of the above-mentioned excipients. Controlled slow-release formulations are also preferred, including osmotic pumps and layered delivery systems.

Liquid dosage forms for oral administration include pharmaceutically acceptable emulsions, solutions, suspensions, syrups, and elixirs. In addition to the Indolium compounds, the liquid dosage forms may contain inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethylene glycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents.

Suspensions may contain suspending agents, as for example, ethoxylated iso-stearyl alcohols, polyoxyethylene sorbitol and sorbitan esters, microcrystalline cellulose, aluminum metahydroxide, bentonite agar-agar and tragacanth, or mixtures of these substances, and the like.

Compositions for rectal administrations are preferably suppositories which can be prepared by mixing the compounds of the present disclosure with suitable nonirritating excipients or carriers such as cocoa butter, polyethylene glycol or a suppository wax, which are solid at ordinary temperatures but liquid at body temperature and therefore, melt in the rectum or vaginal cavity and release the active component.

Dosage forms for topical administration include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this disclosure.

Pharmaceutical compositions typically comprise an effective amount of Indolium compounds disclosed herein and a suitable pharmaceutical acceptable carrier. The preparations can be prepared in a manner known per se, which usually involves mixing the at least one compound according to the disclosure with the one or more pharmaceutically acceptable carriers, and, if desired, in combination with other pharmaceutical active compounds, when necessary, under aseptic conditions. Reference is made to U.S. Pat. Nos. 6,372,778, 6,369,086, 6,369,087 and 6,372,733 and the further references mentioned above, as well as to the standard handbooks, such as the latest edition of Remington's Pharmaceutical Sciences. It is well known that ester prodrugs are readily degraded in the body to release the corresponding alcohol. See e.g., Imai, Drug Metab Pharmacokinet. (2006) 21(3):173-85, entitled “Human carboxylesterase isozymes: catalytic properties and rational drug design.

The pharmaceutical preparations of the disclosure are preferably in a unit dosage form, and can be suitably packaged, for example in a box, blister, vial, bottle, sachet, ampoule or in any other suitable single-dose or multi-dose holder or container (which can be properly labeled); optionally with one or more leaflets containing product information and/or instructions for use. Generally, such unit dosages will contain between 1 and 1000 mg, and usually between 5 and 500 mg, of the at least one compound of the disclosure e.g., about 10, 25, 50, 100, 200, 300 or 400 mg per unit dosage.

### Indolium 1 Exerts Activity against Vemurafenib Resistant Melanoma in Vivo

The development of targeted therapies (Braf/MEK inhibitors) and immunotherapy have had a major impact on the treatment of melanoma. However, the majority of patients with advanced melanomas succumb to their disease. A triphenylmethane, Indolium 1 has been synthesized, and it demonstrates efficacy against an aggressive vemurafenib resistant melanoma in vivo. Indolium 1 has a novel mechanism of action against melanoma, in that it results in induction of the tumor suppressor EphA3.

The ability to inhibit the growth of vemurafenib resistant melanoma in vivo is unique. Indolium 1 has potent in vivo activity against LM36R which is an aggressive BRAF mutant melanoma that is vemurafenib resistant. Indolium 1 is well tolerated in mice and given its efficacy against vemurafenib resistant melanoma, and it is believed to works through a mechanism independent of BRAF. ATAC-seq reveals that Indolium 1 induces EphA3, which is a tumor neoantigen and tumor suppressor gene.

In order to investigate Indolium 1 activity against LM36R, vemurafenib-resistant melanoma cell line, in vivo, 10 mice were inoculated at 1×10⁶ cells/mouse. Mice were then treated with indolium 1 via IP administration three times a week at a concentration of 3 mg/kg/week. Results suggests significant reduction of tumor volume in Indolium 1-treated mice compared to control. (FIG. 2).

To gain insights into the mechanisms by which Indolium 1 caused decrease in LM36R tumor volume, we analyzed possible changes in chromatin structure in the tumor cells using ATAC-seq. EphA3 was one of the most upregulated genes in terms of chromatin accessibility. Immunohistochemical staining results were consistent with this finding, as Indolium 1 treated tumor tissue samples had more positivity for EPHA3 compared to controls. Furthermore, immunohistochemical staining for Nkx3.2 also showed upregulated Nkx3.2 expression in Indolium-treated cells compared to control cells. To further explore the effects that Indolium 1 has on cells which could have led to tumor size reduction, LM36R cells were treated with varying concentrations of Indolium 1. Subsequently, it was found that pMAPK and pAkt levels tended to with Indolium 1 treatment, while levels of Rb decreased with increasing concentrations of Indolium 1 treatment. Beta-tubulin was used to confirm even protein concentrations in each sample. Immunohistochemical staining for Rb showed results consistent with the Western blot, with Indolium 1 treated tumor tissue expressing less Rb compared with control tissue.

The induction of MAPK and Akt seem to contribute to reduction in tumor size observed in mice treated with Indolium 1. In a cell viability assay, cells were treated with Indolium 1, MEK inhibitor (inhibiting the MAPK signaling pathway directly upstream of MAPK), and Akt inhibitor. Cells were also treated with a combination of Indolium 1 and each inhibitor. Co-treatment with MEK inhibitor inhibited cell death, as cell viability was significantly higher in the Indolium 1 and MEK inhibitor group compared to Indolium 1 alone. The same effect was observed when cells were co-treated with Akt inhibitor and Indolium 1 (FIG. 3).

Experiments reported herein indicate that small molecule, Indolium 1 has activity against vemurafenib resistant BRAF mutant melanoma. Vehicle versus Indolium treated tumors in mice were analyzed for potential biomarkers and for insight into the mechanism of Indolium 1 in vivo. One of the most upregulated genes in terms of chromatin accessibility is EphA3, which has relevance in melanoma biology. EphA3 is a receptor tyrosine kinase which binds ligands ephrinA5 and IL-26. While tumor stimulatory and inhibitory functions have been ascribed to EphA3, the predominance of the evidence suggest that EphA3 is a tumor suppressor. First, inactivating mutations in EphA3 are often found in high grade malignancies. Second, activation of wild type EphA3 causes downregulation of Akt, which has previously been implicated in melanoma progression. Third, EphA3 has been shown to be a tumor neoantigen in melanoma. Finally, a novel antibody based therapeutic directed against EphA3, and therapies that induce EphA3 could sensitize melanoma to anit-EphA3 antibodies.

Surprisingly, Indolium 1 causes the loss of expression of the pRb tumor suppressor gene. This might be seen as a negative for an antitumor agent, but more recent studies demonstrate that there are oncogenic functions of pRb. Cells lacking pRB, but not Rb homologs p107 and p130, have greatly increased resistance to transformation with oncogenic Ras, compared to isogenic wild type fibroblasts. Complete loss of pRb is mostly confined to retinoblastoma, small cell carcinoma of the lung, and osteosarcoma. In contrast, high level expression of pRB is associated with a poor prognosis in melanoma. Karim et al. report reduced p16 and increased cyclin D1 and pRb expression are correlated with progression in cutaneous melanocytic tumors. Int J Surg Pathol 2009, 17, 361-367. Chicas et al. report the unique role of the retinoblastoma tumor suppressor during cellular senescence. Cancer Cell 2010, 17, 376-387. The loss of pRb expression is noted on both Western blot analysis and by immunohistochemistry of treated tumors. This provides a biomarker activity of Indolium 1.

The treatment of LM36R melanoma cells also leads to a surprising induction of phosphorylated MAP kinase and Akt. This could be a protective response to Indolium 1 expression, or a requirement for Indolium 1 induced cell death. In order to determine these possibilities, LM36R cells were treated in the presence of inhibitors of MAPK, and Akt to determine whether these inhibitors would protect or potentiate cell death due to In-dolium 1. Both inhibitors provided protection against Indolium 1 induced cell death. This has important potential clinical implications. First, resistance to targeted therapies is associated with re-expression of MAP kinase and Akt, so Indolium 1 would be well positioned to treat melanoma which has recurred after targeted therapies. In certain embodiments, this disclosure contemplates the use of BRAF/MEK inhibitors with Indolium 1 in a sequential fashion.

### Synthesis of Indolium 1

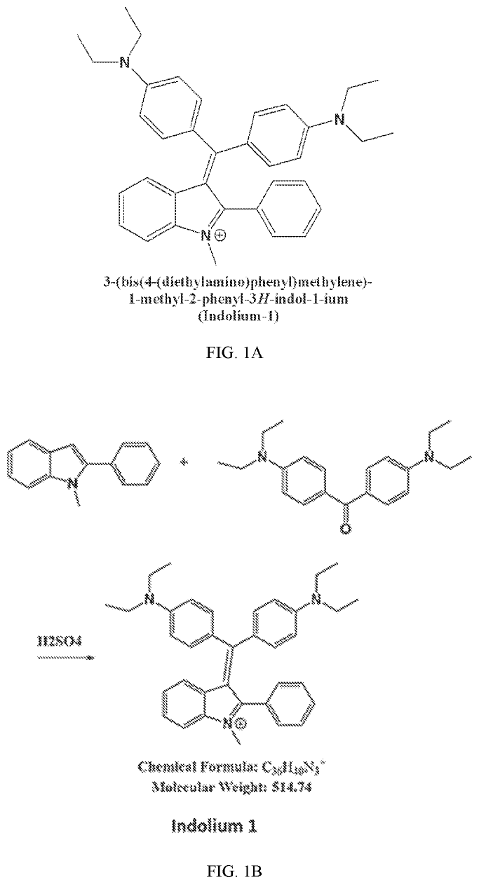

Ethyl Mishler's ketone (1 gram, CAS: 90-93-7), of and 1-methyl-2-phenylindole (0.68 grams, CAS: 3558-24-5) were heated with 0.2 mL sulfuric acid at 75° C. until the mixture turned blue (FIG. 1). Methanol (10 ml) of was added, and heated briefly to 75° C. After 1 hour, methanol was added to a total volume of 50 mL, was neutralized with of sodium carbonate (0.2 grams), and excess Michlers ketone was extracted with hexane until the hexane layer was no longer yellow. Reaction was then purified by liquid extraction and column chromatography. Purity and structure were assessed by thin-layer chromatography (TLC), liquid chromatography-mass spectrometry (LC-MS), and nuclear magnetic resonance (NMR).

### Proliferation Assay

A cell proliferation assay was used to study the effect of Indolium 1 in vitro. In a 96 well plate, 50,000 LM36 cells were incubated in 6 lanes (4 wells in each lane) with 200 μL/well of the aforementioned growth media. After 24 hours, the media was changed to treated media. Lane 1 served as a control with DMSO added. Lane 2 was treated with Indolium 1 (1 μM) (stock solution: 5.14 mg/mL dissolved in DMSO). Lanes 3 and 4 were treated with 10 μM MEK inhibitor U0126 (stock solution: 1 mg/mL in DMSO) and 10 μM Calbiochem Akt Inhibitor II (Product #124008, Sigma-Aldrich) (stock solution: 1 mg/mL in DMSO). Lane 5 was treated with a combination of Indolium 1 (1 μM) and MEK inhibitor U0126 (10 μM), while Lane 6 was treated with a combination of Indolium 1 (1 μM) and Akt inhibitor II (10 μM). Cells were incubated for 24 hours. 20 μL of AlamarBlue™ cell viability reagent was added to each well. After 4 hours, the plate was read following manufacturer's instructions and the data was processed using Softmax™ Pro software (Molecular Devices). An unpaired two-tailed Student's t-test was performed to determine the significant difference between the control and each treatment group, along with between the Indolium 1 only versus Indolium 1 plus MEK/Akt inhibitor, with significance determined at p<0.05.

### Western Blotting

LM36R cells were grown in six T75 flasks. Upon reaching 70% confluence, cell media was replaced with media with concentrations of 0, 0.1, 0.5, 1, 2.5, and 5 μM Indolium 1. After 24 hours, cells were lysed in RIPA buffer (25 mM Tris-HCl, 150 mM NaCl, 1% NP-40, 1% sodium deoxycholate, 0.1% sodium dodecyl sulfate, pH 7.6) supplemented with HALT™ protease phosphatase inhibitor cocktails and 0.5 M EDTA solution. Cell lysates incubated on ice for 30 minutes, and then centrifuged at 16,000×g at 4° C. for 20 minutes. Protein concentration was determined using the Pierce BCA Protein Assay Kit™ and then normalized. NuPage™ LDS Sample Buffer (4×) and beta-mercaptoethanol were added to the samples, which were subsequently boiled. Sample protein (40 μg) of were loaded into each well of NuPAGE™ 4-12% Bis-Tris precast gels in MOPS buffer against Precision Plus™ Protein Dual Color Standards. Proteins were transferred onto polyvinylidene difluoride membrane using Transblot Turbo™ system. Membrane was blocked for 1 hour at room temperature in 5% non-fat dry milk in 0.1% Tween-Tris-Buffered Saline and probed with Rb (#PA5-27215) pMAPK (9101S), pAkt (#4060S), and beta-tubulin (#2146S) antibodies at 1:1000 dilutions in 5% bovine serum albumin at 4° C. overnight. The membranes were then incubated in HRP-linked anti-rabbit IgG secondary antibody (#7074S) with a 1:1000 dilution for 1 hour at room temperature. Antibody signal was detected using SuperSignal™ West Pico chemiluminescence substrate with Medical X-Ray Film and digitally scanned.

### In Vivo LM36R Xenograft Model

The xenograft model was developed. A LM36R cell suspension in growth medium was inoculated at 1×10⁶ cells/mouse in the right flank of athymic Nu/Nu nude male mice (n=5 per group). MPIB was prepared by dissolving 3.75 mg into 1 mL of ethanol 7. This solution (100 μL) was then diluted to the final concentration in 1 mL of 20% soy-fat Intralipid™ prior to each injection and vortexed vigorously. Vehicle control (ethanol in Intralipid) or MPIB was administered intraperitoneally three times a week at 3 mg/kg/week. MPIB treatment was initiated on the second day after the tumor cell injection, and the tumor volume as well as the weight of the animals were recorded weekly thereafter. Animals were sacrificed and tumors were harvested after tumor volumes reached experimental endpoint as determined by IACUC protocol.

### Immunohistochemistry of in Vivo LM36R Tumor Model

Tumor samples were resected and embedded in formalin, processed, and stained. Formalin-fixed and paraffin-embedded tissue sections from each group were cut to a 5-μm thickness and air-dried. Processing was performed using Ventana DISCOVERY™ Ultra automated immunohistochemistry stainer. Slides were deparaffinized with EZ-Prep (#05279771001) and then were antigen retrieved for 64 minutes with CC1 reagent (#950-500). Rabbit anti-Eph receptor A3 (EphA3) antibody (ab126261) diluted at 1:50, Rabbit anti-Nkx3.2 (#PA5-21108 diluted at 1:500) and mouse anti-Rbl antibody (554136) diluted at 1:100 were applied and incubated for 40 minutes. For the first two stains, DISCOVERY™ OmniMap anti-Rb HRP was applied and incubated for 12 minutes, and the detection was completed in combination with DISCOVERY™ ChromoMap DAB kit for anti-EphA3 antibody and DISCOVERY™ Red kit for anti-Nkx3.2 anti-body, as per manufacturer recommendations. For the Rbl stain, DISCOVERY™ OmniMap anti-mouse HRP was applied and incubated for 12 minutes, and the detection was completed in combination with DISCOVERY™ ChromoMap DAB kit. Slides were counterstained with hematoxylin for 8 minutes. Slides were then dehydrated, cover-slipped, and evaluated by light microscopy. Whole imaging was performed.

1. A method of treating or preventing cancer comprising administering an effective amount of a compound having the following formula: 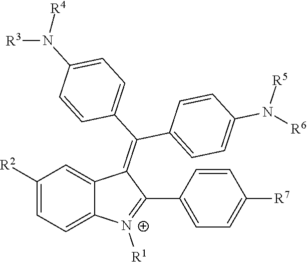
or salt thereof wherein,
R¹, R², R³, R⁴, R⁵, R⁶, and R7, are individually and independently selected from hydrogen, halogen, alkyl, hydroxy, alkoxy, mercapto, alkylthio, amino, alkylamino, dialkylamino, carboxyl, nitro, and nitrile,
to a subject in need thereof, optionally in combination with another anticancer agent.

2. The method of claim 1 wherein the compound is a 3-(bis(4-(diethylamino)phenyl) methylene)-1-methyl-2-phenyl-3H-indol-1-ium (Indolium-1) salt.

3. The method of claim 1 wherein the cancer is melanoma, uveal melanoma, acral melanoma, melanoma mutant in NRAS, or triple negative melanoma.

4. The method of claim 1 wherein the compound is administered in combination with another anticancer agent.

5. The method of claim 1 wherein the anticancer agent is an inhibitor of v-Raf murine sarcoma viral oncogene homolog B (BRAF) such as vemurafenib, dabrafenib, and encorafenib.

6. The method of claim 1 wherein the anticancer agent is an inhibitor of mitogen-activated protein kinase kinase (MEK) such as trametinib, binimetinib, selumetinib, and cobimetinib.

7. The method of claim 1 wherein the anticancer agent is an inhibitor of or an inhibitor of protein kinase B (AKT) such as ipatasertib.

8. The method of claim 1 wherein the anticancer agent is an anti-CTLA4 antibody such as ipilimumab and tremelimumab.

9. The method of claim 1 wherein the anticancer agent is an anti-PD1 antibody such as nivolumab, pembrolizumab, and cemiplimab.

10. The method of claim 1 wherein the anticancer agent is an anti-PD-L1 antibody such as atezolizumab, avelumab, and durvalumab.

11. The method of claim 1 wherein the cancer is selected from bladder cancer, lung cancer, breast cancer, colon cancer, rectal cancer, endometrial cancer, pancreatic cancer, kidney cancer, prostate cancer, thyroid cancer, brain cancer, multiple myeloma, lymphoma, or leukemia.

12. A pharmaceutical composition comprising a compound having the following formula: 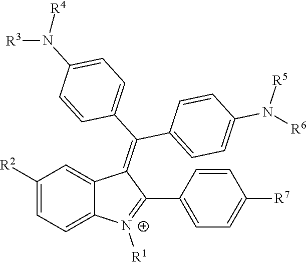
or salt wherein,
R¹, R², R³, R⁴, R⁵, R⁶, and R⁷, are individually and independently selected from hydrogen, halogen, alkyl, hydroxy, alkoxy, mercapto, alkylthio, amino, alkylamino, dialkylamino, carboxyl, nitro, and nitrile,
and a pharmaceutically acceptable excipient.

13. The pharmaceutical composition of claim 12 wherein the compound is 3-(bis(4-(diethylamino) phenyl) methylene)-1-methyl-2-phenyl-3H-indol-1-ium (Indolium-1) salt.

14. The pharmaceutical composition of claim 12 in the form of a lotion, gel, cream, pill, tablet, capsule, or gel capsule.

15. The pharmaceutical composition of claim 12 in the form of a sterilized pH buffered aqueous salt solution or a saline phosphate buffer between a pH of 6 to 8, optionally comprising a saccharide or polysaccharide.
